SEQUENCE LISTING (4 sequences)

SEQ ID NO: 1 - protein, 137 aa, Homo sapiens
MSSLIRRVISTAKAPGAIGPYSQAVLVDRTIYISGQIGMDPSSGQLVSGGVAEEAKQALKNMGEILKAAGCDFTNVVKTTVLLADINDFNTVNEIYKQYFKSNFPARAAYQVAALPKGSRIEIEAVAIQGPLTTASL

SEQ ID NO: 2 - protein, 135 aa, Mus musculus
MSSIIRKVISTTKAPAAIGPYSQAVQVDRTIYISGQVGLDPSSGQLVPGGVVEEAKQALKNLGEILKAAGCDFNNVVKTTVLLADMNDFGTVNEIYKTYFQGSLPARAAYQVAALPRGSRVEIEAIAVQGPFIKA

SEQ ID NO: 3 - protein, 104 aa, Mus musculus
PYSQAVQVDRTIYISGQVGLDPSSGQLVPGGVVEEAKQALKNLGEILKAAGCDFNNVVKTTVLLADMNDFGTVNEIYKTYFQGSLPARAAYQVAALPRGSRVEI

SEQ ID NO: 4 - protein, 14 aa, synthetic construct
HHHHHHLEVLFQGP

(19) United States
(12) Patent Application Publication
(10) Pub. No.: US 2026/0258391 A1
(43) Pub. Date: Sep. 3, 2026

(54) REACTIVE INTERMEDIATE DEAMINASE A (RIDA) FOR USE IN PREVENTING AND/OR TREATING CACHEXIA

(71) Applicants: Giovanni BUSSOLATI, Torino (IT); Benedetta BUSSOLATI, Torino (IT)

(72) Inventors: Giovanni BUSSOLATI, Torino (IT); Benedetta BUSSOLATI, Torino (IT)

(21) Appl. No.: 19/163,961

(22) PCT Filed: Mar. 14, 2024
(86) PCT No.: PCT/IB2024/052448

§ 371 Date: Sep. 10, 2025

(30) Foreign Application Priority Data
Mar. 14, 2023 (IT) 102023000004776

(51) Int. Cl. C12N 9/78; A61K 38/00; A61P 35/00

(52) U.S. Cl. CPC C12N 9/78; A61P 35/00; A61K 38/00; C12Y 305/99

(57) ABSTRACT

The present invention relates to reactive intermediate deaminase A (ridA), or functionally active fragment thereof, or composition comprising said deaminase or said fragment for use in preventing and/or treating cachexia, preferably neoplastic cachexia, or in inhibiting tumour growth in an individual suffering from tumours, wherein the deaminase is homologous of the individual.

### CROSS-REFERENCE TO RELATED APPLICATIONS

This patent application claims priority from Italian Patent Application No. 102023000004776 filed on Mar. 14, 2023, the entire disclosure of which is incorporated herein by reference.

### TECHNICAL FIELD

The present invention relates to a reactive intermediate deaminase A (ridA), or functionally active fragment thereof, or composition comprising said deaminase or said fragment for use in preventing and/or treating cachexia, preferably neoplastic cachexia, or in inhibiting tumour growth in an individual suffering from tumours.

### BACKGROUND OF THE INVENTION

The term ‘cachexia’ refers complex systemic metabolic syndrome which commonly affects individuals with malignant neoplasms, particularly late stage, and is manifested by a depletion of muscle mass. Although the main cause of cachexia is the development of neoplastic forms, cachexia can also be induced by chronic heart failure, renal failure and auto-immune diseases.

Such a syndrome is accompanied by widespread malaise, commonly referred to as ‘fatigue,’ and negatively impacts the quality of life, survival and ability to tolerate treatment in cancer patients, in particular the cytotoxic chemotherapy they must undergo. Although, therefore, skeletal muscle atrophy is the most obvious manifestation of neoplastic cachexia, the latter is currently considered a systemic phenomenon affecting various organs and apparatuses and including fatigue and neoplastic pain.

The pathogenesis of neoplastic cachexia and associated fatigue is unknown and no specific treatments are available. Such a lack of valid therapeutic strategies has stimulated research into the mechanisms behind the phenomenon, and numerous studies have suggested that the possible causes lie in toxic metabolites produced by the neoplasm as consequence of the rapid cell proliferation associated therewith. However, the precise nature of these toxic metabolites is currently unknown. Experimentally, the most widely used model to study neoplastic cachexia is the C26 model in BALB/C mice, and a significant serum marker has been identified in interleukin-6 (IL-6).

The rapid proliferative growth characterising the cells of malignant neoplasms involves increased protein synthesis and amino acid supply, in particular serine. Studies conducted by Downs and collaborators focused on the possibility that accelerated serine synthesis leads to overproduction of a toxic intermediate metabolite, 2-amino-acrylate (2 AA). Produced by serine metabolism, 2AA is relatively unstable, but can exert a harmful toxic effect by forming adducts with pyrodoxal-5-phosphate (PLP), resulting in the blockade of PLP-dependent enzymes, in particular dehydratase and deaminase.

Numerous studies, in particular on microbial organisms, indicate that the accumulation of 2AA and the subsequent metabolic damage to PLP-dependent enzymes is controlled and prevented by RidA (reactive intermediate e deaminase A) proteins, which facilitate the hydration of enamine, resulting in the formation of a ketone product so as to avoid cell damage. The RidA proteins (YjgF/YER057c/UK114/heat responsive protein 12, HRSP12/P14.5/PSP, 14.5 KDa translational inhibitor protein/ribonuclease UK114) are all members of a super-family of archetypal proteins conserved from microbes to mammals, which act by controlling the accumulation of 2AA and preventing subsequent damage to PLP-dependent enzymes which would lead to a cellular metabolic block. The enzymatic activity in RidA proteins is linked to the formation of a ‘pocket’ designed to capture the ligand (2AA), defined by the 3D arrangement of 7 amino acids whose cadenced sequence arrangement characterises this precise family. These are the amino acids which in some species have the following cadence: Y-17, S-30, G-31, N-56, N-88, R-105, P-114, E-120 and the substitution of such amino acids, in particular the substitution of R (arginine) 105 with Alanine causes the loss of function. Since the amino acid sequences in the initial part of the molecule can vary, the reference number also varies, but not the cadenced sequence. In mouse RID-A, as well as in human RID-A, R results in position 107. The positions of the 7 amino acids in human and mouse RID-A appear to be in the position: Y-21, S-34, G-35, N-61, N-93, R-107, P-116, E-122.

PLP-dependent enzymes such as decarboxylases and aminotransferases play a central role in the metabolism of gamma-amino butyric acid (GABA), and on the other hand it is known that blocking GABA-ergic neurotransmission leads to muscle atrophy and chronic pain perception.

The mammalian component of the RidA family of proteins is the UK114 protein, originally identified as a fraction soluble in perchloric acid, which was subsequently sequenced and whose immino-deaminase activity on 2AA was recently demonstrated. The antigenic characteristics of this protein have made it possible to demonstrate its presence in tumour cells and, in particular, on the cell surface.

In view of the problems that neoplasms and more in particular neoplastic cachexia, as well as cachexia more in general, entail for the individual and society, there is a need in the art to provide new molecules for preventing and/or treating cachexia, preferably neoplastic cachexia, and for inhibiting tumour growth.

It is an aim of the present invention to provide a molecule or composition capable of preventing and/or treating cachexia, preferably neoplastic cachexia, the pain and physical wasting associated therewith, and of inhibiting tumour growth.

According to the present invention, such an aim is achieved by the use of a reactive intermediate deaminase A (ridA), or functionally active fragment thereof or composition comprising said deaminase or said fragment as defined in claim 1.

### BRIEF DESCRIPTION OF THE FIGURES

For a better understanding of the present invention, it will also be described with reference to the attached figures, which illustrate the following:

FIG. 1 is a graph showing the effects of the treatment on neoplastic cachexia with homologous (mouse) and heterologous (human) RidA in the C26 (carcinoma cells) mouse model;

FIG. 2 illustrates three graphs showing the effects of the treatment with homologous (mouse) and heterologous (human) RidA on liver, fat and spleen weight respectively in the C26 model;

FIG. 3 is a graph showing the effects on IL-6 serum levels of the treatment with homologous (mouse) and heterologous (human) RidA in the C26 model;

FIG. 4 is a graph showing the effects of the treatment with homologous (mouse) and heterologous (human) RidA on C26 tumour sizes and on T-lymphocyte infiltration, as assessed by (FACS) cytofluximetry and anti-CD3 antibodies;

FIG. 5 is a graph showing the effects on neoplastic cachexia of the treatment with homologous (mouse) RidA administered subcutaneously and orally in the C26 (carcinoma cells) mouse model;

FIG. 6 is a graph showing the effects on neoplastic cachexia with reference to liver, spleen, fat and muscle respectively of the treatment with homologous (mouse) RidA administered subcutaneously and orally in the C26 model (carcinoma cells) in mice;

FIG. 7 is a graph showing the effects of tumour volume reduction following treatment with homologous (mouse) RidA administered subcutaneously and orally in the C26 (carcinoma cells) mouse model;

FIG. 8 is a graph showing the effects of treatment with mouse RidA (RIDA-M) and peptide comprising 104 aa of mouse RidA protein (mouse RIDA-M 20-123) in the C26 model, in particular on the weight of the animal;

FIG. 9 is a graph showing the effects of the treatment with mouse RidA (RIDA-M) and peptide comprising 104 aa of mouse RidA protein (mouse RIDA-M 20-123) in the C26 model, in particular on the IL6 serum level;

FIG. 10 is a graph showing the effects of the treatment with mouse RidA (RIDA-M) and peptide comprising 104 aa of mouse RidA protein (mouse RIDA-M 20-123) in the C26 model, in particular on tumour weight;

FIG. 11 is a graph showing the effects of treatment with mouse RidA (RIDA-M) and peptide comprising 104 aa of mouse RidA protein (mouse RIDA-M 20-123) in the C26 model, in particular on muscle and fat weight;

FIG. 12 is a graph showing the deaminase activity of semicarbazone by mouse RidA (RIDA-M) and mouse RidA with arginine mutation in position 107 (RIDA-M R107) over time;

FIG. 13 is a graph showing the Interleukin 6 (IL6) serum level, a sensitive indicator of neoplastic cachexia, in: normal mice, tumour-carrying mice, tumour-carrying mice treated with homologous RidA (RIDA-M) and tumour-carrying mice treated with homologous RidA mutated in amino acid 107 (RIDA-M-R107).

### DETAILED DESCRIPTION OF THE INVENTION

According to the present invention, a reactive intermediate deaminase A (ridA), or functionally active fragment thereof or composition comprising said deaminase or said fragment is used in preventing and/or treating cachexia, preferably neoplastic cachexia, in an individual. The deaminase is preferably homologous of the individual. Said deaminase, said fragment or said composition is also used, according to the present invention, to inhibit tumour growth in an individual suffering from tumours.

The deaminase is preferably UK114/HRSP12, even more preferably human UK114/HRSP12. UK114/HRSP12 human has sequence SEQ ID NO: 1. Deaminase function is also maintained by proteins with sequences with a certain percentage of homology, e.g., with homology of at least 90%, and by protein fragments. In fact, it has been observed that, in mice, having a UK114/HSRP12 sequence SEQ ID NO: 2 (135aa), a 104 aa fragment of the RidA protein, comprising the 7 amino acids characteristic of RidA proteins (mouse RidA 20-123) and having SEQ ID NO: 3 (PYSQAVQVDRTIYI SGQVGLDPSSGQLVPGGVVEEAKQALKNLGEILKAAGCDENNVV KTTVLLADMNDFGTVNEIYKTYFQGSLPARAAYQVAALPRGSRVEI—the 7 amino acids mentioned above in bold), produced results similar to the complete mouse RidA protein.

The deaminase, fragment or composition is preferably administered subcutaneously, intramuscularly, by nasal or sublingual route.

Preferably, said deaminase, said fragment or said composition facilitate the action of at least one immunotherapeutic drug. Examples of such treatments are the use of CAR-T (chimeric antigen receptor modified T cells).

Preferably, said deaminase, said fragment or said composition reduces tumour-associated pain in the individual.

The term ‘fragment’ or ‘functionally active fragment’ refers to a fragment of deaminase which maintains the enzymatic function of immino-deamination. In particular, fragments retaining 7 amino acids characteristic of RidA proteins have been found to retain this function. An example of a functionally active fragment in mice is a 104 aa fragment of the RidA protein, comprising the 7 amino acids characteristic of RidA proteins (mouse RidA 20-123) and having SEQ ID NO: 3.

In the following examples, the RidA protein is shown to have a role in treating neoplastic cachexia. This role was experimentally tested using a recombinant protein and a recognised experimental mouse model of neoplastic cachexia.

By virtue of these experiments, it was deduced that the toxic metabolite responsible for neoplastic cachexia, fatigue and pain associated with tumour development is 2-amino-acrylate (2AA), and that such a toxic metabolite acts by blocking PLP-dependent enzymes, in particular decarboxylases and aminotransferases involved in GABA metabolism, resulting in muscle atrophy and pain.

Furthermore, it has been observed that the RidA family protein expressed in mammals exerts immine-deamidase enzymatic activity, preventing the accumulation of 2AA. The RidA protein therefore exerts an active role r in treating neoplastic cachexia and in the induced pain.

Again, it has been shown that in the case of prevention and/or treatment of cachexia, the deaminase need not be homologous of the individual. In fact, all UK114/HRSP12/RID proteins are characterised by the presence of the sequence of 7 aa highlighted in bold above in SEQ ID NO: 3. The modification of only one of these amino acids results in the loss of specific enzyme activity and thus the activity of the UK114/HRSP12 proteins (Lambrecht et al., The Journal of biological chemistry, 287 (5): 3454-3461, 2012). It has been shown that the anti-cachexia activity is linked to the presence of the aforesaid 7 amino acids and the resulting enzymatic activity of the protein, so much so that by modifying only one amino acid at the specific R107 site, both the enzymatic activity and anti-cachexia action are lost. Examples

Mouse protein UK 114, a 135 amino acid protein (SEQ ID NO: 2: MSSIIRKVISTTKAPAAIGPYSQAVQVDRTIYI SGQVGLDPSSGQLVPGGVVEEAKOAL KNLGEILKAAGCDFNNVVKTTVLLADMNDFGTVNEIYKTYFQGSLPARAAYQVAALPRG SRVEIEAIAVQGPFIKA) was produced by the company Sino Biological Inc. (No. 18 Kechuang 10th Street Building 9, Beijing, P. R. China, 100176) following the following protocol: 1) plasmid synthesis; 2) infection of E. coli and induction of protein synthesis, 3) column purification of the protein 4) protein control on SDS-PAGE.

For the purpose of column protein separation and purification, a recognition site (His-tag) and separation site (3C site-tortoise) was applied to the protein sequence: HHHHHHLEVLFQGP (SEQ ID NO: 4).

A sample of human RidA Protein (Ribonuclease UK114/HRSP12 Protein Human (His) Cat. No.: HY-P70372) produced by MCE (MedChemExpress) (NJ 08852, USA) was purchased by D.B.A. italia (Milan, Italy).

A 104 central aa fragment of the mouse RidA protein, extended from aa. 20 to aa. 123, including the 7 amino acids characteristic of RidA proteins, namely a fragment (mouse RidA 20-123) with SEQ ID NO:3: PYSQAVQVDRTIYISGQVGLDPSSGQLVPGGVVEEAKOALKNLGEI LKAAGCDENNVVK TTVLLADMNDFGTVNEIYKTYFQGSLPARAAYQVAALPRGSRVEI (the 7 amino acids in bold), was produced by the company LifeTein (LLC. 601 US Rt. 206, Suite 26-463 Hillsborough, NJ 08844, U.S.A.)

The correctness of the synthesised mouse protein was checked by western blot technique, testing it against an anti-RIDA serum produced in rabbits (anti-HRSP12) by the company Prestige Antibodies®Powered by Atlas Antibodies) and purchased from the company Sigma (Milan, Italy).

The C-26 model in BALB/C mice was used as cachexia model. This model is commonly used in studies of neoplastic cachexia, which was performed according to the following guidelines: subcutaneous implantation in the dorsal region in 7-week-old BALB/C mice. In each animal, 1×10⁶ in vitro cultured cells of the C26 line (a clone of a colon carcinoma arising in BAL/C mice treated with N-nitroso-N-methyl urethane) were implanted. The tumour developed rapidly and the mice were sacrificed after 3 weeks. The animal carcasses were weighed. The carcinomas were measured and harvested for histological examination (after fixation in Phosphate buffered Formalin and inclusion in paraffin) and immunohistochemical examination. The tumour cells were also separated and a cytofluorometric examination was carried out on the cell suspension for the presence and percentage of T lymphocytes (identified with antibody anti-CD3). The skeletal muscles, in particular the gastrocnemius Soleus and Tibialis muscles of the hind legs, were taken, weighed frozen by immersion in liquid Nitrogen. Cryostat sections, oriented orthogonally to the fibre direction, were obtained with a Leica cryostat and processed for immunohistochemical staining for laminin antigen. Sections were also stained with haematoxin-eosin, scanned with Aperio Scanner and the transverse diameter of the fibres measured using ImageJ.

Blood was obtained from the sacrificed animals to assess IL-6 by ELISA (R&D systems).

The data were statistically analysed (Anova) and reported as **=P<0.001 vs CTL and *=P<0.05 vs CTL.

The data obtained show that the RidA protein, endowed with immino-deamidase activity linked to the amino-acid sequence specific to RidA proteins, is capable of reducing or eliminating the accumulation of toxic substances (in particular 2AA) induced by neoplastic growth, thus resulting in the reduction, prevention or cancellation of induced cachexia phenomena. The results were obtained using the homologous (mouse) RidA protein. Similar, though slightly inferior, results were obtained with heterologous (human) protein. This shows that, by analogy, optimal results should be expected in humans using the administration of RidA Homo in patients. The sequence of such a protein (RidA Human: 137 aa: MSSLIRRVIS TAKAPGAIGP YSQAVLVDRT IYISGQIGMD PSSGOLVSGG VAEEAKQALK NMGEILKAAG CDFTNVVKTT VLLADINDEN TVNEIYKQYF KSNFPARAAY QVAALPKGSR IEIEAVAIQG PLTTASL; SEQ ID NO: 1) shows similarities with that of mice, with an identity percentage of 81%. In human patients, it is expected that treatment with human RidA may also reduce, prevent or reverse the phenomena associated with cachexia, in particular neoplastic cachexia, such as fatigue or chronic pain.

A reduction in C26 tumour volume was also observed following treatment with RidA protein, demonstrating the properties of the RidA protein, in particular homologous RidA protein (mouse, in mice and consequently human protein in patients) in the treatment of tumours. In tumours from animals treated with homologous RidA protein, an increase in intratumoural CD3-positive (cytotoxic) lymphocytes was also demonstrated alongside the reduction in tumour volume. This finding suggests that the treatment with homologous RidA protein may promote intra-tumour lymphocyte infiltration and the immunotherapy of tumours. In fact, it is well known that the intra-tumour lymphocyte infiltrate is an indicative parameter of the body's anti-tumour response.

It was also observed that a 104 aa. fragment of the mouse RidA protein, including the 7 amino acids characteristic of RidA proteins (mouse RidA 20-123), and specifically a fragment with SEQ ID NO: 3: PYSQAVQVDRTIYISGQVGLDPSSGQLVPGGVVEEAKQALKNLGEILKAAGCDENNVVK TTVLLADMNDFGTVNEI YKTYFQGSLPARAAYQVAALPRGSRVEI (7 amino acids in bold), produced results similar to the complete mouse RidA protein.

### Example 1

The experiment was carried out on 19 BALB/C mice. Four animals were kept as controls, while the others were injected subcutaneously, dorsally, with the same amount of C26 carcinoma cells. In two batches of animals, treatment was carried out with subcutaneous injection of mouse RidA (supplied by Sino Biologicals) (MS UK) and human RidA (supplied by MCE). In both cases, the RidA was administered at a dose of 2.5 mg/kg. On days 1, 7, 14 and 21, the animals were weighed and the tumour volume measured by calibre. The injections were carried out on days 1, 7 and 14, while on day 21 the animals were sacrificed, as planned in the design. A 5% reduction in animal weight was considered as an indication of neoplastic cachexia.

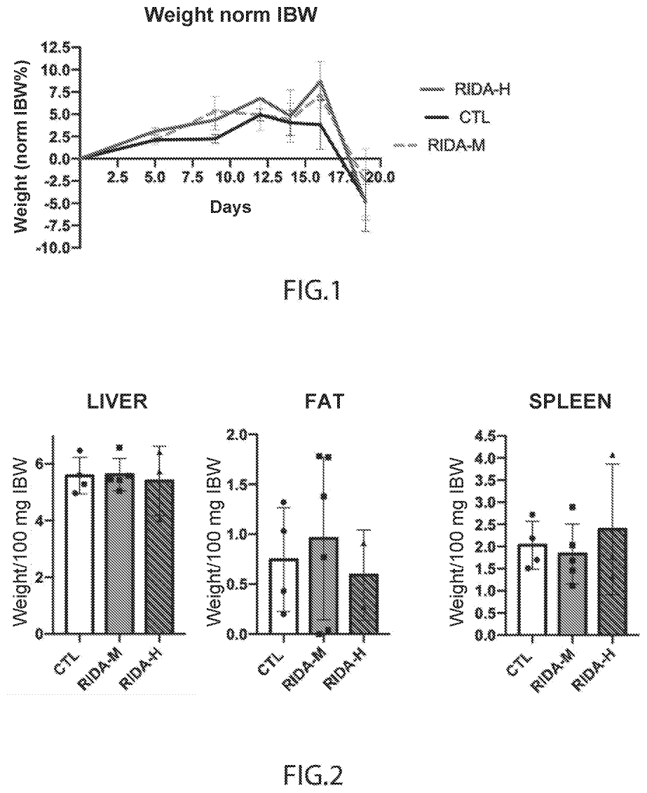

As can be seen in FIG. 1, both treatments (with homologous RidA (mouse, RIDA-M) and with heterologous RidA (human, RIDA-H)) showed a significant effect in reducing neoplastic cachexia, as evidenced by the lower weight reduction of the animals. The weight reduction of the animals was not statistically significant between homologous for heterologous RidA.

FIG. 2 shows that the reduction in weight of the various organs was not statistically significant between the control and treated, except for fat weight, which was less reduced in the animals treated with homologous RidA.

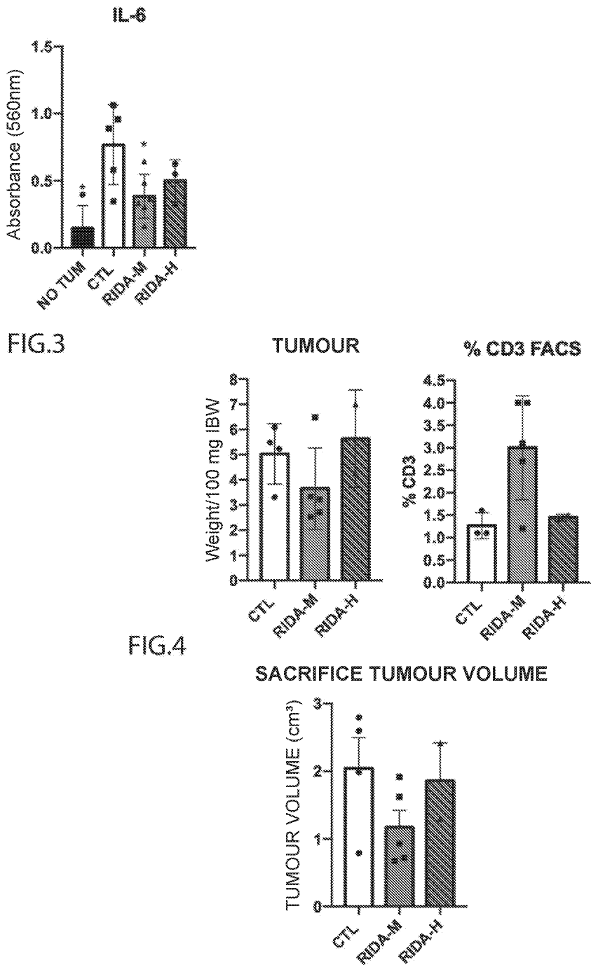

On the other hand, FIG. 3 shows how the interleukin-6 (IL-6) serum level, considered in the literature to be a sensitive indicator of neoplastic cachexia, was significantly reduced with respect to the controls in both homologous and heterologous RidA-treated mice. However, the reduction was significantly greater in the animals treated with homologous RidA.

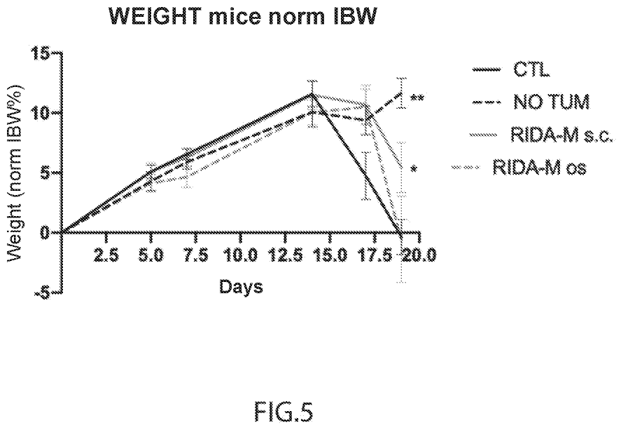

In the experiment, the effects of treatment with homologous (mouse) and heterologous (human) RidA on C26 tumour size and T-lymphocyte infiltration, as assessed by cytofluximetry (FACS) and anti-CD3 antibodies, were also checked. A significant effect of the treatment with homologous (mouse) RidA was noted with a reduction in neoplastic mass, correlated with a specific increase in cytotoxic T-lymphocyte infiltrate. The reduction of the neoplastic mass was particularly noticeable on day 14 of the experiment (FIG. 5).

### Example 2

The experiment was carried out on 19 BALB/C mice. Four animals were kept as controls, while the others were injected subcutaneously, dorsally, with the same amount of C26 carcinoma cells. In one batch of animals, the treatment was carried out by subcutaneous injection of mouse RidA (supplied by Sino Biologicals) (MS UK), administered at a dose of 2.5 mg/kg by weight, while in the other batch a double dose (5 mg/kg by weight) was administered by gavage. On days 1, 7, 14 and 21, the animals were weighed and the tumour volume measured by calibre. The injections and oral administrations were carried out on days 1, 7 and 14, while on day 21 the animals were sacrificed, as planned in the design. A 5% reduction in animal weight was considered as an indication of neoplastic cachexia.

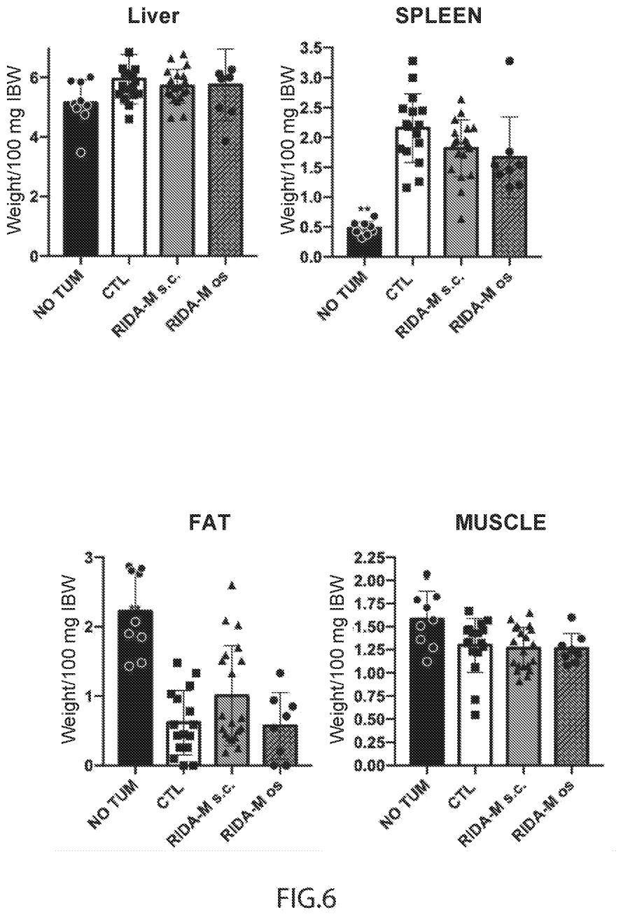

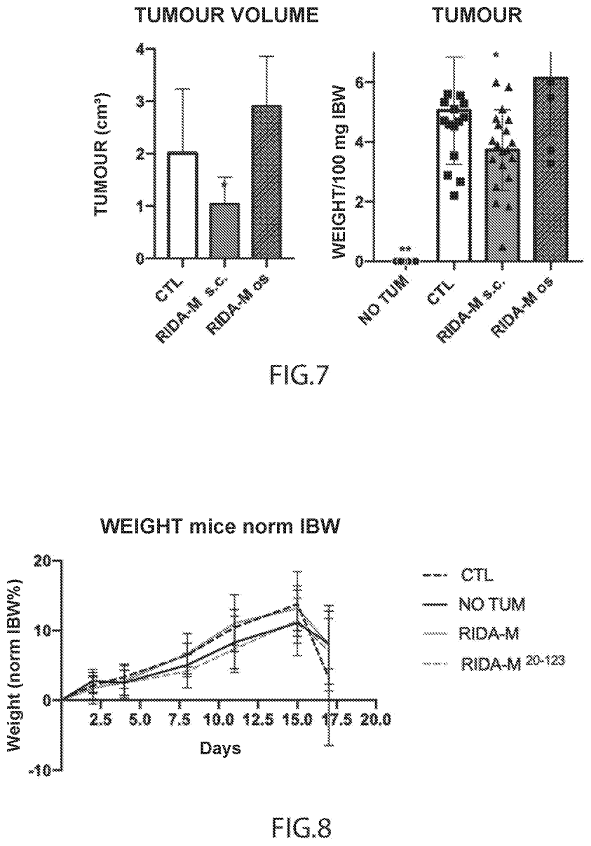

The treatment with homologous (mouse) RidA gave a significant effect in reducing neoplastic cachexia, as demonstrated by the lower weight reduction of the animals (FIG. 5), thus confirming the data obtained in example 1. In contrast, the anti-cachexia effect as well as the neoplastic mass reduction effect are absent in animals treated with RidA but administered orally, demonstrating that the anti-cachexia activity as well as the tumour volume reduction effect are related to enzyme activity and disappear after ingestion and relative protein digestion (FIG. 6 and FIG. 7).

### Example 3

The experiment was carried out on 19 BALB/C mice. Four animals were kept as controls, while the others were injected subcutaneously, dorsally, with the same amount of C26 carcinoma cells. In two batches of animals, treatment was carried out with subcutaneous injection of mouse RidA (supplied by Sino Biologicals) (RIDA-M) and mouse RIDA-M 20-123 (manufactured by LifeTein). In both cases, the RidA was administered at a dose of 2.5 mg/kg. On days 1, 7, 14 and 21, the animals were weighed and the tumour volume measured by calibre. The injections were carried out on days 1, 7 and 14, while on day 21 the animals were sacrificed, as planned in the design. A 5% reduction in animal weight was considered as an indication of neoplastic cachexia.

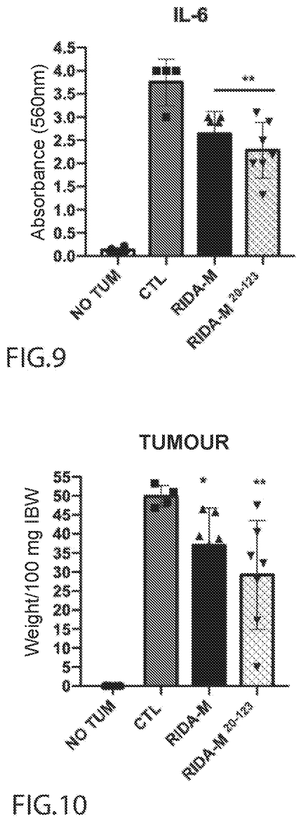

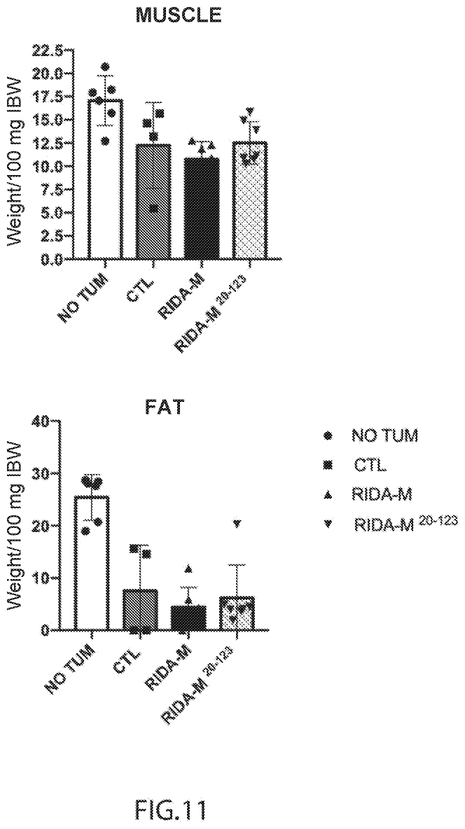

In order to check whether the effect of the RidA in counteracting neoplastic cachexia is due to the intact protein alone, or whether a similar effect can be obtained with a central protein fragment comprising the 7 amino acids characterising Rid function, we carried out an experiment on C26 tumour-carrying mice, according to the above scheme. The experiment (see FIG. 8) demonstrates that even a protein fragment, as long as it includes the 7 aa mentioned above, is capable of exerting activity counteracting C26 tumour-induced cachexia. similar results were Substantially obtained with regard to the weight of the animal (FIG. 8), and with regard to serum IL6 values (FIG. 9) and the downsizing of the C26 tumour (FIG. 10) upon animal sacrifice.

### Example 4

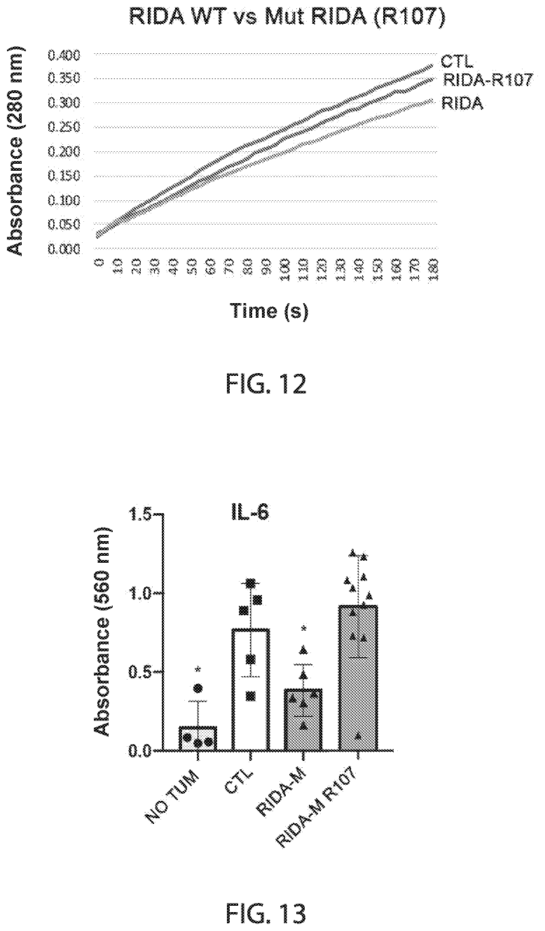

In this example, we show how the described effects of the homologous RidA protein on tumours, and in particular on the tumour-induced ‘cachexia’ effect (reduction in animal weight loss and less tumour atrophy, as detectable by the smaller reduction in the diameter of muscle fibres) are the specific amino-imidasic directly correlated with activity of the mouse RidA protein. The modification of a single amino acid (R107) in the 136 amino acid sequence of the RidA protein (RIDA-M R107), such an amino acid being a constituent part of the ‘pocket’ designated for enzymatic activity (Lambrecht et al, 2012) along with 6 other amino acids defining the RidA and UK114 proteins, not only results in a substantial loss of enzyme activity, as illustrated in FIG. 12, but is also related to a loss of tumour volume reduction and cachexia activity, as observed in the whole protein.

Similarly, treatment in tumour-carrying mice with mutated protein (RIDA-M R107) is not capable of reducing the serum level of Interleukin-6 (IL-6), as is instead induced by the whole molecule (RIDA-M), an effect characteristically associated with the reduction of cachexia, as illustrated in FIG. 13.

These data confirm that the anti-cachexia effect of homologous and heterologous RIDA proteins is linked to the imidasic enzymatic activity with detoxification of 2-amino-acrylate (2AA), an activity related to the maintenance of the constitutive sequence of the 7 amino acids defining the belonging of the protein to the RIDA series.

1. Reactive intermediate deaminase A (ridA), or functionally active fragment thereof or composition comprising said deaminase or said fragment for use in preventing and/or treating of cachexia, preferably neoplastic cachexia, in an individual.

2. Reactive intermediate deaminase A (ridA), or functionally active fragment thereof or composition comprising said deaminase or said fragment for use according to claim 1, wherein the deaminase is homologous of the individual.

3. Reactive intermediate deaminase A (ridA), or functionally active fragment thereof or composition comprising said deaminase or said fragment for use in inhibiting tumour growth in an individual suffering from tumour, wherein the deaminase is homologous of the individual.

4. Reactive intermediate deaminase A (ridA), or functionally active fragment thereof or composition comprising said deaminase or said fragment for use according to claim 1, wherein the deaminase is UK114/HRSP12.

5. Reactive intermediate deaminase A (ridA), or functionally active fragment thereof or composition comprising said deaminase or said fragment for use according to any of claim 1, wherein the deaminase is human UK114/HRSP12.

6. Reactive intermediate deaminase A (ridA), or functionally active fragment thereof or composition comprising said deaminase or said fragment for use according to claim 5, wherein the deaminase has a sequence SEQ ID NO:1.

7. Reactive intermediate deaminase A (ridA), or functionally active fragment thereof or composition comprising said deaminase or said fragment for use according to claim 1, wherein said deaminase, said fragment or said composition is administered subcutaneously, intramuscularly, by nasal or sublingual route.

8. Reactive intermediate deaminase A (ridA), or functionally active fragment thereof or composition comprising said deaminase or said fragment for use according to claim 1, wherein said deaminase, said fragment or said composition promote the action of at least one immunotherapeutic drug.

9. Reactive intermediate deaminase A (ridA), or functionally active fragment thereof or composition comprising said deaminase or said fragment for use according to claim 1, wherein said deaminase, said fragment or said composition reduce the pain associated to a tumour in an individual.
